(19) United States
(12) Patent Application Publication
(10) Pub. No.: US 2022/0250931 A1
(43) Pub. Date: Aug. 11, 2022

(54) Method for Oxidizing Copper Chloride II Using Electromagnetic Induction

(71) Applicant: Billy Zachery Earley, Corona, CA (US)

(72) Inventor: Billy Zachery Earley, Corona, CA (US)

(21) Appl. No.: 17/169,656

(22) Filed: Feb. 8, 2021

(51) Int. Cl. C01G 3/05; D03D 15/283; D03D 9/00; D01F 1/10; H05B 6/10

(52) U.S. Cl. CPC C01G 3/05; D03D 15/283; D03D 9/00; D01F 1/103; H05B 6/101; A61L 2202/11; A41D 31/30; A41D 2500/20; D10B 2501/04; D10B 2331/04; D10B 2403/0243; D10B 2401/13; D03D 2700/0174

(57) ABSTRACT

An innovative high-energy oxidative method using metallic copper and chlorine liquid to produce a superior copper chloride II element using electromagnetic induction and magnetic forces. This invention involves copper undergoing oxidation while in its highest energy state according to basic principles of electromotive forces described in Faraday's law. The copper attaches to a magnetic receptacle and held in place by a copper lid cover. Research studies demonstrate that metallic copper is not magnetic; however, when a magnetic field approaches copper, the electrons and subatomic particles forms a higher resistance against the magnets—Generating a force field response towards the approaching magnets. The oxidation of copper in its highest energy state provides additional improvements and benefits in copper's antimicrobial and antiviral properties. This new method for oxidizing copper chloride in its highest subatomic energy state provides vast improvements and coverage in the fight against microorganisms and the invisible pathogens abroad.

### TECHNICAL FIELD

This invention relates to oxidation and reduction of metallic copper element combined with chlorine in liquid form to produce copper chloride II using electromagnetic induction. The use of electromagnetic induction permits the copper electrons and subatomic particles to undergo oxidation while in its highest energy state, thus, creating a high-energy oxidation method for producing copper chloride II. The application of copper chloride II is used in hospitals and public settings to fight against microbial pathogens and safety.

### BACKGROUND

The use of Electromagnetic Induction to oxidize copper chloride was invented to help in the fight against the invisible pathogens and viral species. Recent studies in the U.S. demonstrate that wasteful healthcare spending alone is estimated to reach nearly 935 billion dollars by 2021. In addition, one out of every 25 people in the United States will get an accidental infection from a hospital and or visiting their healthcare providers. The production of a high-energy copper chloride element can be used to store and protect personal property equipment (PPE), examination beds, medical clothing, protective gloves, and using a high-energy copper chloride component will provide superior safety for millions of people requiring mask mandates around the world.

Research study shows that metallic copper is 99.9% effective against mostly all microorganisms and viral species using 1000 parts per million, which is a 20 to 1 ratio calculating H₂O drops compared to grains of copper. In order to yield similar antimicrobial efficacy, the electromagnetic oxidation of copper chloride requires a minimum conversion factor from parts per million converted to milligrams per cubic centimeters, at a ratio of 28 to 1 (28.0 Mg/Cm³).

The oxidation of copper using electromagnetic induction can occur using any amount of metallic copper, however; a minimum of 28.0 milligrams of copper per cubic centimeters is required in order to maintain a 99.9% efficacy against the invisible pathogens and viral species. In addition, metallic copper oxidation using electromagnetic induction can be oxidized using any form, such as: solids, sheets, wire, pipes, tubing, and copper can be easily oxidized when produced with fabrics and polyester threads.

Presently, there is no system or invention on Earth that can safely store/protect PPEs during or after work hours and millions of healthcare providers are storing their medical tools in very dangerous places. For example, PPEs are being stored in the trunk of cars, in back seats, in glove compartment boxes, at work in or on the desk, in dirty lockers, backpacks, in closets at home, and other dangerous places.

Therefore, a need exists for method for oxidizing copper chloride II using electromagnetic induction that protect human life and provide greater safety against the invisible pathogens and viral species in general. The present invention attempts to address the limitations and deficiencies of present solutions according to the principles and example embodiments disclosed herein. In particular, a method using Electromagnetic Induction to create a high-energy system for copper oxidation leading to the production of copper chloride II using electromagnetic forces. The application and construction of the copper chloride II invention is designed to decrease accidental transmission and delivery of deadly microorganisms and viral pathogens in healthcare environments and public domains. The construction of this newly formed copper chloride II invention can be applied to many products in order to minimize possible exposure and transmission of pathogens.

### SUMMARY

In accordance with the present invention, the above and other problems are solved by providing a method for oxidizing copper chloride II using electromagnetic induction according to the principles and example embodiments disclosed herein.

In another embodiment, the present invention is a method for oxidizing copper chloride II using electromagnetic induction. The method attaches a copper component to a magnetic receptacle using a copper lid, submerges the magnetic and copper component receptacle in boiling water at 212 degrees Fahrenheit for 5 minutes, submerges the magnetic receptacle and copper component into a liquid chlorine solution for 2 minutes, submerges the magnetic receptacle and copper component into warm water at 120 degrees Fahrenheit, and submerges the magnetic receptacle with the copper component into a beaker with cold water for 2 minutes creating copper chloride II material.

In another aspect of the present disclosure, the copper chloride II material being created using electromagnetic induction at 28.0 milligrams of copper chloride II per cubic centimeters (28.0 Mg/Cm³).

In another aspect of the present disclosure, the copper chloride II material comprises one of the following: copper chloride II meshwork, solid copper chloride II material, copper chloride II pipe, copper chloride II tubing, and copper chloride II sheet metal.

In another aspect of the present disclosure, the copper chloride II material comprises copper and polyester threading material woven into a copper chloride II meshwork

In another aspect of the present disclosure, the copper chloride II meshwork using electromagnetic induction being used to manufacture lab coats, nursing tops, pants, masks, gloves, storage cases, and examination bed tops.

The foregoing has outlined rather broadly the features and technical advantages of the present invention in order that the detailed description of the invention that follows may be better understood. Additional features and advantages of the invention will be described hereinafter that form the subject of the claims of the invention.

It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features that are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only, and is not intended as a definition of the limits of the present invention.

### BRIEF DESCRIPTION OF THE DRAWINGS

Referring now to the drawings in which like reference numbers represent corresponding parts throughout:

FIG. 1 depicts a frontal view diagram of a copper atom during its resting state depicting the copper nucleus, subshells, and electrons.

FIG. 2 depicts a frontal view diagram of FIG. 1 with a super imposed layer depicting the copper electrons energy changes when approaching an electromagnetic field.

FIG. 3 depicts a frontal view of a diagram of a copper wire mesh network combined with a polyester fabric material.

FIGS. 4A-B depict a diagram of the 5 steps required to oxidize and form the production of copper chloride II using electromagnetic induction.

FIG. 5 depicts a frontal view of a diagram of a lab coat and the copper chloride II mesh network forming the pockets to protect the PPE's stored inside the pockets.

FIG. 6 depicts a frontal view of a diagram depicting a nursing scrub top with the pockets constructed with the copper chloride II mesh network.

FIG. 7 depicts a frontal view of a diagram depicting a pair of men pants with the pockets constructed with copper chloride II.

FIG. 8 depicts a frontal view of a diagram of a facial mask made with the construction of copper chloride II.

FIG. 9 depicts a frontal view of a diagram depicting a pair of gloves constructed with copper chloride II.

FIG. 10 depicts a frontal view of a diagram depicting a storage case container for storing medical tools constructed from copper chloride II lining the upper and lower cases.

FIG. 11 depicts a frontal view of a diagram depicting a medical examination bed.

### DETAILED DESCRIPTION

This application relates a system and method for oxidizing copper chloride II using electromagnetic induction according to the present invention according to the present invention.

Various embodiments of the present invention will be described in detail with reference to the drawings, wherein like reference numerals represent like parts and assemblies throughout the several views. Reference to various embodiments does not limit the scope of the invention, which is limited only by the scope of the claims attached hereto. Additionally, any examples set forth in this specification are not intended to be limiting and merely set forth some of the many possible embodiments for the claimed invention.

In describing embodiments of the present invention, the following terminology will be used. The singular forms “a,” “an,” and “the” include plural referents unless the context clearly dictates otherwise. Thus, for example, reference to “a needle” includes reference to one or more of such needles and “etching” includes one or more of such steps. As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. As used herein, the singular forms “a,” “an,” and “the” are intended to include the plural forms as well, unless the context clearly indicates otherwise.

It further will be understood that the terms “comprises,” “comprising,” “includes,” and “including” specify the presence of stated features, steps or components, but do not preclude the presence or addition of one or more other features, steps or components. It also should be noted that in some alternative implementations, the functions and acts noted may occur out of the order noted in the figures. For example, two figures shown in succession may in fact be executed substantially concurrently or may sometimes be executed in the reverse order, depending upon the functionality and acts involved.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “50-250 micrometers” should be interpreted to include not only the explicitly recited values of about 50 micrometers and 250 micrometers, but also include individual values and sub-ranges within the indicated range. Thus, included in this numerical range are individual values such as 60, 70, and 80 micrometers, and sub-ranges such as from 50-100 micrometers, from 100-200, and from 100-250 micrometers, etc.

Unless otherwise indicated, all numbers expressing quantities of ingredients, properties such as molecular weight, percent, ratio, reaction conditions, and so forth used in the specification and claims are to be understood as being modified in all instances by the term “about,” whether or not the term “about” is present. Accordingly, unless indicated to the contrary, the numerical parameters set forth in the specifications and claims are approximations that may vary depending upon the desired properties sought to be obtained by the present disclosure. At the very least, and not as an attempt to limit the application of the doctrine of equivalents to the scope of the claims, each numerical parameter should be construed in light of the number of reported significant digits and by applying ordinary rounding techniques. Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in the testing measurements.

As used herein, the term “about” means that dimensions, sizes, formulations, parameters, shapes, and other quantities and characteristics are not and need not be exact, but may be approximated and/or larger or smaller, as desired, reflecting tolerances, conversion factors, rounding off, measurement error and the like, and other factors known to those of skill. Further, unless otherwise stated, the term “about” shall expressly include “exactly,” consistent with the discussion above regarding ranges and numerical data.

The term “invention” or “present invention” refers to the invention being applied for via the patent application with the title “Method for oxidizing copper chloride II using electromagnetic induction.” Invention may be used interchangeably with copper chloride II.

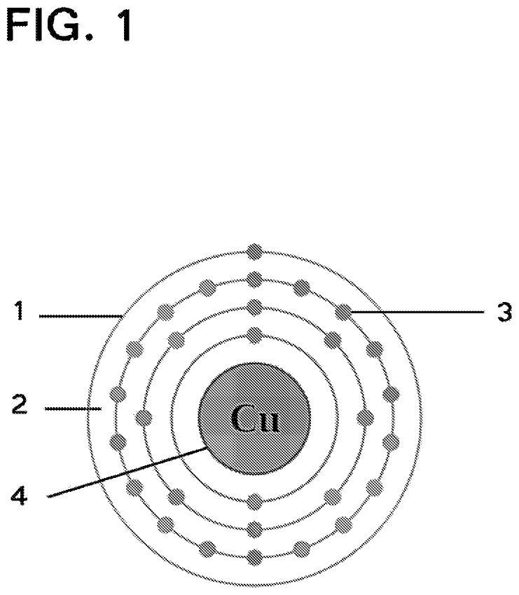

In general, the present disclosure relates a system and method for oxidizing copper chloride II using electromagnetic induction. FIG. 1, now referring to the drawing in FIGS. 1-11, wherein similar components are identified by like numerals, there is seen in FIG. 1 depicts a frontal view drawing of copper atom diagram 1 showing its configuration having four subshells 2 and each subshell comprise electrons 3 that circulates around the nucleus 4 of the copper atom.

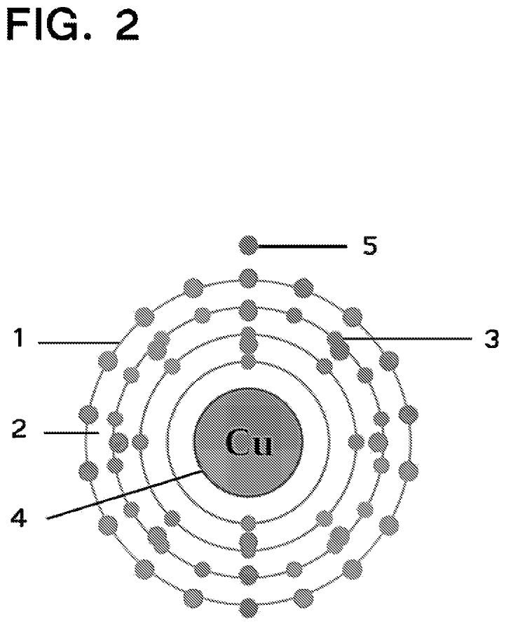

FIG. 2, now referring to the drawing in FIG. 2, depicts the copper atom 1 and the diagram in FIG. 1 depicting the electrons 3 changing from a normal resting state of the copper atom 1 to a high-energy subatomic state 5 reaching the upper subshells 2 of the copper atom 1. The copper resting energy state increases as the magnetic field approaches towards the copper elements.

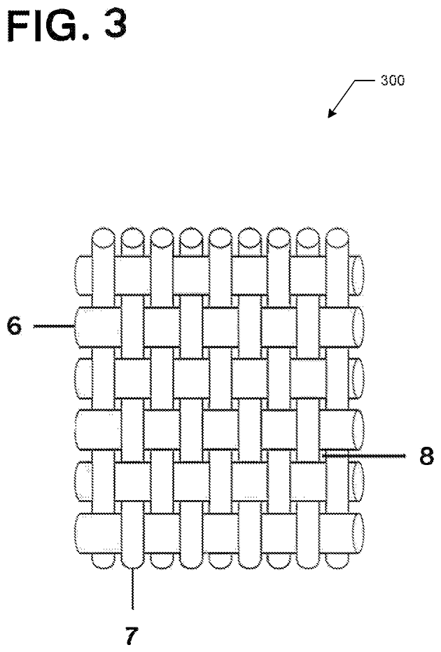

FIG. 3, now referring to the drawings in FIG. 3, depicts a mesh network combing copper filament 6 sewn and weaved together with a polyester material 7 forming a copper polyester meshwork with porous fields 8. Copper chloride II material, as disclosed herein, may be fashioned into the copper filament 6 that may be woven into a meshwork 300. The copper chloride II meshwork 300 can be applied to any pocket design, briefcase, clothing, equipment covering, or any surface requiring protection against microorganisms and viral pathogens in general.

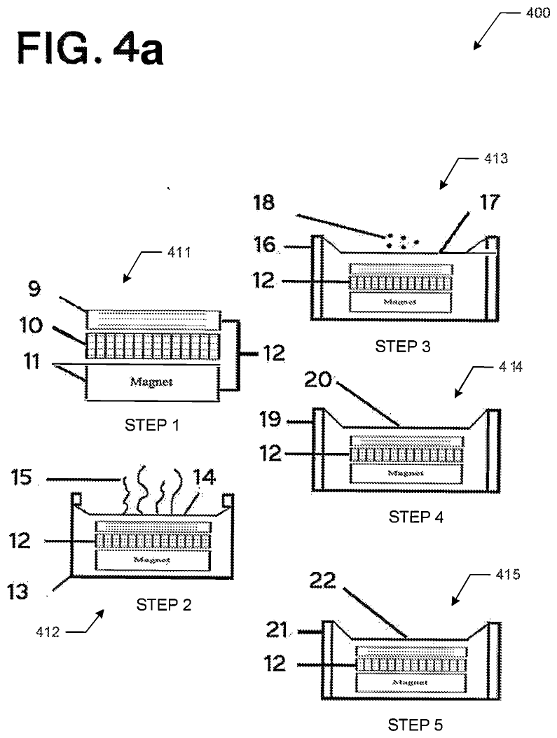

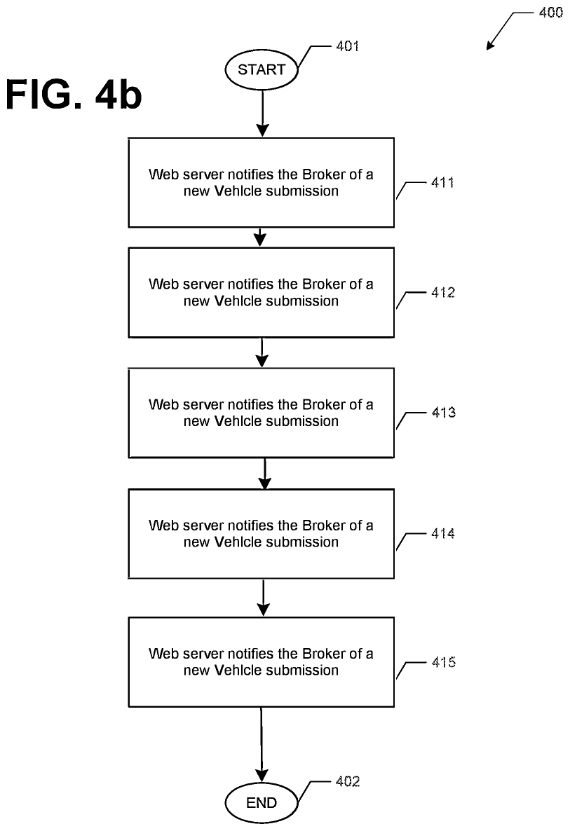

FIGS. 4a-b, now referring to the drawings in FIG. 4a-b, is diagrams depicting steps 1-5 for producing a copper chloride II high-energy oxidation reaction using Electromagnetic Induction. FIG. 4a shows the configuration of the components within the device manufacturing the copper chloride II meshwork. FIG. 4b shows a flowchart depicting the steps performed in the method 400 according to the present invention. The process 400 begins with copper meshwork 10 placed on top of a magnetic receptacle 11, in step 411, and attached with a copper lid 9. The magnetic receptacle 11, lid 9, and the copper meshwork 10 forms a secure holding assembly 12 to secure the copper chloride method. In step 412, the assembly 12 is submerged into a beaker 13 with boiling water 15 at 212 degrees Fahrenheit for 5 minutes. This is a preparatory step to increase permeability of the copper and chloride II reaction and oxidation process.

The heated copper and magnetic receptacle assembly 12 is submerged, in step 413, into a beaker 16 containing chlorine liquid producing a rapid reaction between the copper meshwork 10 and the chlorine 17 producing steam and small particles of chlorine gas 18 to escape. The copper magnetic receptacle assembly 12 is left inside the chlorine liquid 17 for 2 minutes to oxidize and reduce the copper chlorine to copper chloride II.

The magnetic receptacle and copper assembly 12 is submerged in beaker 19 contain hot water 20 at 120 degrees Fahrenheit for 2 minutes in step 414. The process concludes with step 415 depicts the magnetic receptacle assembly 12 submerged in beaker 21 with cold water 22 for 2 minutes. The copper element/meshwork 10 is removed from the magnetic holding receptacle 12 resulting in a high-energy oxidation reaction using Electromagnetic Induction. The copper chloride II meshwork 300 has been created may now be used in various items to line pockets and containers to create storage locations that provide a pathogen free location for PPEs.

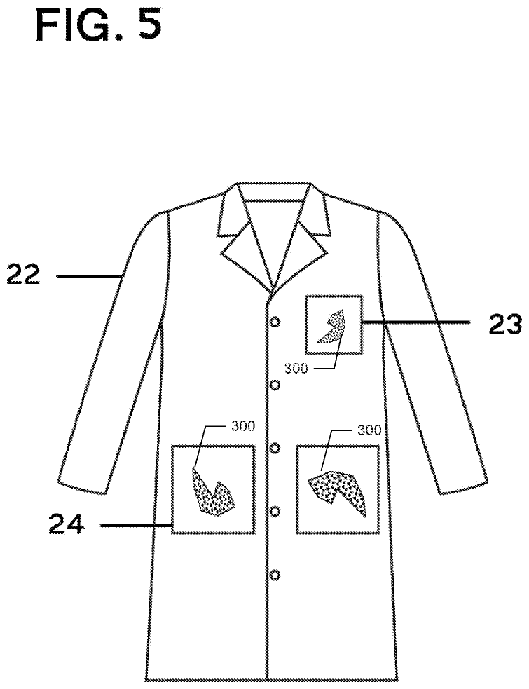

FIG. 5, now referring to the drawing in FIG. 5, depicts a frontal view diagram of a lab coat 22 used by doctors, nurses, and healthcare workers. The copper chloride II meshwork 300 and seen in FIG. 3 is used to form the lab coat pockets 23 and pockets to hold/store PPEs 24. The production and improvements of lab coat 22 pockets 23, 24 with an electromagnetic induced high-energy copper chloride II meshwork 300 will help to sterilize and sanitize PPE's and other tools stored in the pockets 23, 24.

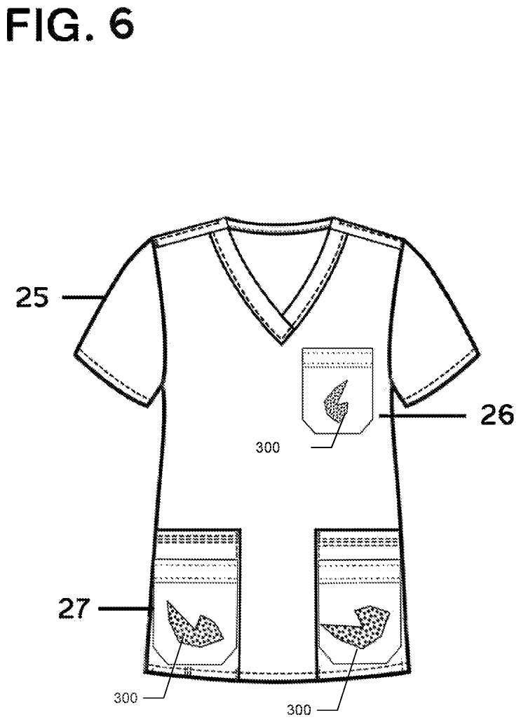

FIG. 6, now referring to the drawing in FIG. 6, depicts a frontal view diagram of a nursing scrub top 25 depicting an upper pocket 26 and two larger lower pockets 27. The diagram depicts the copper chloride II meshwork 300 forming the nursing scrub 25 pockets. Nurses and their PPE's are exposed to many dangerous pathogens in their work settings and research study shows these pathogens are commonly transferred to patients and to other healthcare providers. The copper chloride II meshwork 300 protects against the invisible pathogens and improves safety within the nursing industry.

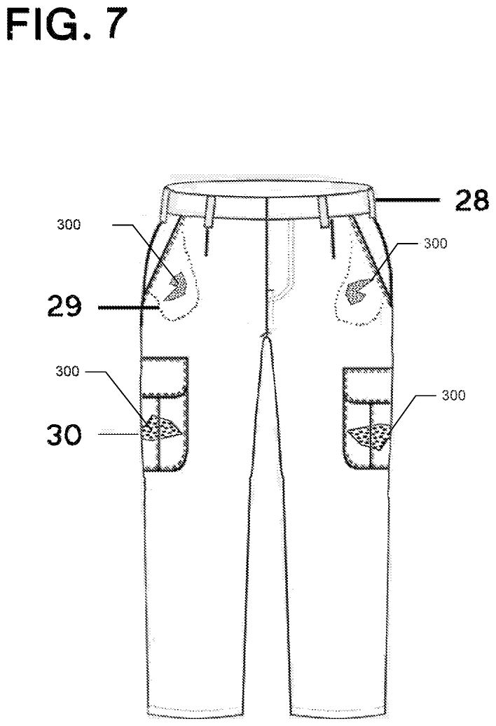

FIG. 7, now referring to the drawing in FIG. 7, depicts a frontal view diagram of a pair of men pants 28 with two upper pockets 29 and two lower pockets 30 produced with the copper chloride II meshwork 300. The improvements in pocket safety against microorganisms and viral pathogens will help to reduce and limit the transmission and spread of microbial infections across many platforms.

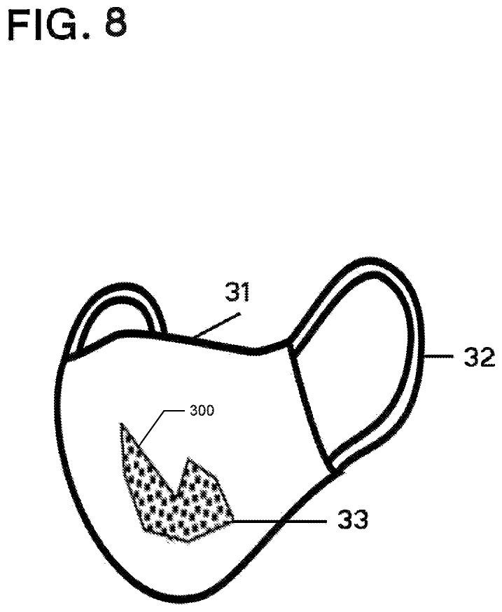

FIG. 8, now referring to the drawing in FIG. 8, depicts a frontal view diagram of a facial mask 31 formed using copper chloride II meshwork 300 that comprises an ear strap assembly 32 and a mouth and nose protective covering 33. Copper Chloride II meshwork 300 for facial masks 31 improves safety against viral and bacterial transmission. Copper Chloride II kills 99.9% bacterial and viral pathogens on contact and protects the mask against pathogens dwelling on the outer and inner mask surfaces.

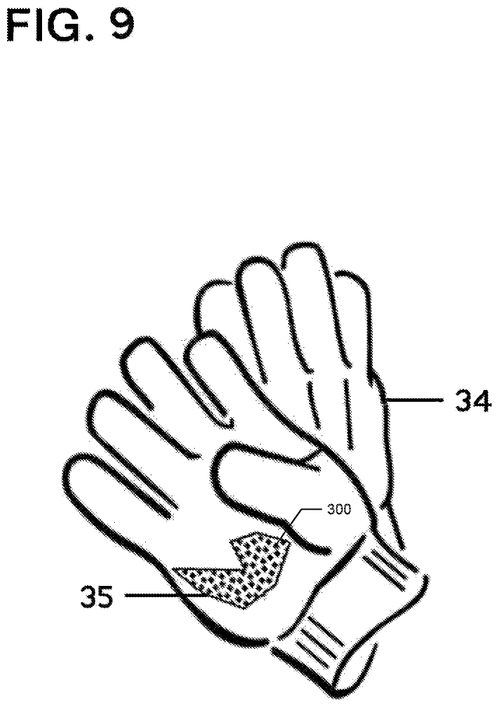

FIG. 9, now referring to the drawing in FIG. 9, depicts a frontal view diagram of a pair of hand gloves 34 produced with copper chloride II meshwork 300 covering the glove body 35. The application of copper chloride II meshwork 300 to glove construction and wear improves safety and hand sanitation naturally. The hands are the number one cause of bacterial and viral transmissions amongst all people according to industry standards.

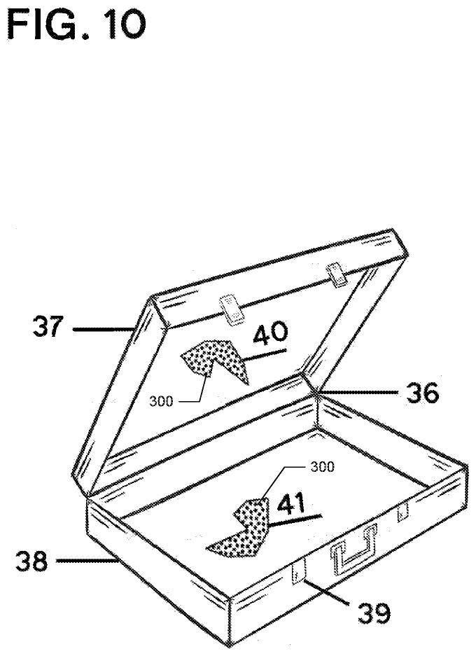

FIG. 10, now referring to the drawing in FIG. 10, depicts a frontal view diagram of a small storage case 36 with an upper 37 and lower 38 case closures and locking device 39. Copper chloride II meshwork 300 seen in FIG. 3 is used to layer the upper-case housing 40 and the lower casing housing 41 that protects the contents within the case from bacterial and viral species, thus, protecting PPE's and other tools.

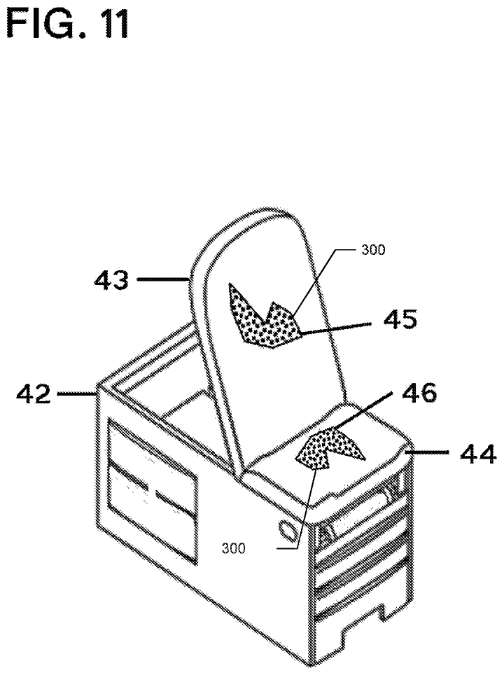

FIG. 11, now referring to the drawing in FIG. 11, depicts a frontal view diagram of an examination bed 42 with a cushion matt covered with copper chloride II meshwork 300 seen in FIG. 3. Most exam beds are sectional with a lower 44 and adjustable upper 43 bed construction. Exam beds use paper rolls to guard against bacterial and viral pathogens and this process is improved greatly by using copper chloride II meshwork 300 with 99.9% protection against microbial surface species to replace upper 45 and lower 46 coverings.

Even though particular combinations of features are recited in the present application, these combinations are not intended to limit the disclosure of the invention. In fact, many of these features may be combined in ways not specifically recited in this application. In other words, any of the features mentioned in this application may be included to this new invention in any combination or combinations to allow the functionality required for the desired operations.

No element, act, or instruction used in the present application should be construed as critical or essential to the invention unless explicitly described as such. Further, the phrase “based on” is intended to mean “based, at least in part, on” unless explicitly stated otherwise. Any singular term used in this present patent application is applicable to its plural form even if the singular form of any term is used.

In the present application, all or any part of the invention's software or application(s) or smart device application(s) may be installed on any of the user's or operator's smart device(s), any server(s) or computer system(s) or web application(s) required to allow communication, control (including but not limited to control of parameters, settings such as for example, sign copy brightness, contrast, ambient light sensor settings . . . etc.), transfer of content(s) or data between any combination of the components

What is claimed is:

1. A method to produce copper chloride II using electromagnetic induction comprises:
attaching a copper component to a magnetic receptacle using a copper lid;
submerging the magnetic and copper component receptacle in boiling water at 212 degrees Fahrenheit for 5 minutes;
submerging the magnetic receptacle and copper component into a liquid chlorine solution for 2 minutes;
submerging the magnetic receptacle and copper component into warm water at 120 degrees Fahrenheit; and
submerging the magnetic receptacle with the copper component into a beaker with cold water for 2 minutes creating copper chloride II material.

2. The method according to claim 1, wherein the copper chloride II material being created using electromagnetic induction at 28.0 milligrams of copper chloride II per cubic centimeters (28.0 Mg/Cm³).

3. The method according to claim 2, wherein the copper chloride II material comprises one of the following: copper chloride II meshwork, solid copper chloride II material, copper chloride II pipe, copper chloride II tubing, and copper chloride II sheet metal.

4. The method according to claim 2, wherein the copper chloride II material comprises copper and polyester threading material woven into a copper chloride II meshwork.

5. The method according to claim 4, wherein the copper chloride II meshwork using electromagnetic induction being used to manufacture lab coats, nursing tops, pants, masks, gloves, storage cases, and examination bed tops.
